(19) United States
(12) Patent Application Publication
(10) Pub. No.: US 2012/0315678 A1
(43) Pub. Date: Dec. 13, 2012

(54) MICROALGA HIGHLY ACCUMULATING STARCH, A METHOD FOR PRODUCING GLUCOSE USING THE SAME, AND A METHOD FOR PRODUCING A TARGET SUBSTANCE

(75) Inventors: Shuhei Hashiro, Kawasaki-shi (JP); Yoshihiro Usuda, Kawasaki-shi (JP)

(73) Assignee: AJINOMOTO CO., INC., Tokyo (JP)

(21) Appl. No.: 13/474,879

(22) Filed: May 18, 2012

(30) Foreign Application Priority Data
May 24, 2011 (JP) 2011-115386

(51) Int. Cl. C12N 1/12; C12P 13/04; C13K 1/00

(52) U.S. Cl. 435/106; 435/257.1; 127/36

(57) ABSTRACT

Glucose is produced by hydrolyzing starch contained in a microalga which belongs to the genus Desmodesmus and accumulates 30% or more of starch in algae bodies based on dry weight of the algae bodies when it is cultured under suitable conditions.

This application claims priority therethrough under 35 U.S.C. §119 to Japanese Patent Application No. 2011-115386, filed May 24, 2011, the entirety of which is incorporated by reference herein. Also, the Sequence Listing filed electronically herewith is hereby incorporated by reference (File name: 2012-05-18T_US-481_Seq_List; File size: 14 KB; Date recorded: May 18, 2012).

### BACKGROUND OF THE INVENTION

1. Field of the Invention

The present invention relates to a novel microalga that highly accumulates starch, and a method for producing glucose using it. Glucose can be used as a raw material for fermentative production of a target substance such as L-amino acids using a microorganism.

2. Brief Description of the Related Art

It is known that green algae, which constitute one class of microalgae, accumulate starch in the cells as a storage polysaccharide. For example, Behrens and P. W. et al., J. Appl. Phycol., 1, 123-130, 1989 describes that a Chlorella vulgaris strain stored 20% of starch based on dry alga body weight in the presence of sufficient nitrogen, and stored 55% of starch based on dry alga body weight in a nitrogen-limited medium. However, any strain showing high starch accumulation rate based on dry alga body weight without using special culture conditions such as nitrogen-limited medium has scarcely been reported.

Hirano, A. et al., Energy, 22, 137-142, 1997 describes that Chlorella vulgaris strain and so forth show 20% or more of starch accumulation rate based on dry alga body weight from oceanic microalgae. However, it has not been previously reported that a green alga belonging to the genus Desmodesmus can highly accumulate starch.

Rodjaroen, S. et al., Kasetsart J., 41, 570-575, 2007 describes that Scenedesmus obliquus belonging to the genus Scenedesmus, which is closely related to the genus Desmodesmus, accumulated 24% of starch based on dry alga body weight. However, the alga body weight of the Scenedesmus obliquus strain obtained by culture over 20 days was 0.3 g/L of the culture medium or less, and thus the productivity based on the unit culture medium volume was low.

It has been reported that glucose can be prepared by using algae that accumulate starch as a raw material, and ethanol fermentation can be performed with that glucose (Japanese Patent Laid-open Nos. 7-31485, 7-87985, 7-87986, 2000-316593, and U.S. Patent Published Application No. 2007/0202582). Furthermore, it has also been reported that ethanol fermentation can be performed by using glucose produced by subjecting algae bodies of a Chlamydomonas reinhardii strain that accumulated starch to a hydrothermal treatment with sulfuric acid (Nguyen, M. T. et al., J. Microbiol. Biotechnol., 19, 161-166, 2009).

Moreover, amino acid fermentation using glucose prepared from starch of a Chlorella vulgaris strain by the alkali treatment method as a raw material has also been reported (International Publication WO2009/093703). However, production of a target substance such as amino acids by fermentation using glucose produced by using a Desmodesmus strain that highly accumulates starch as a raw material has not been reported.

### SUMMARY OF THE INVENTION

An aspect of the present invention is to provide a microalga that highly accumulates starch, a method for producing glucose using it, and a method for producing a target substance such as L-amino acids.

A microalga that highly accumulates starch from water and soil samples is disclosed.

It is an aspect of the present invention to provide a microalga which belongs to the genus Desmodesmus and accumulates 30% or more of starch in algae bodies based on dry weight of the algae bodies when the microalga is cultured under suitable conditions.

It is a further aspect of the present invention to provide the microalga as described above, which can proliferate in a medium not containing vitamin.

It is a further aspect of the present invention to provide the microalga as described above, which accumulates 30% or more of starch in the algae bodies based on dry weight of the algae bodies when the microalga is cultured in a nitrogen non-limited medium.

It is a further aspect of the present invention to provide the microalga as described above, which accumulates 30% or more of starch in algae bodies based on dry weight of the algae bodies when the microalga is cultured at 30° C. for one week in 0.2×Gamborg's B5 medium.

It is a further aspect of the present invention to provide a method for producing glucose, which comprises hydrolyzing starch accumulated in the microalga as described above.

It is a further aspect of the present invention to provide the microalga as described above, which is selected from the group consisting of the strains AJ7835 (FERM BP-11364), AJ7838 (FERM BP-11365) and AJ7840 (FERM BP-11366).

It is a further aspect of the present invention to provide a method for producing a target substance, which comprises culturing a microorganism that produces the target substance in a medium containing glucose produced by the method as described above, and collecting the target substance from culture.

It is a further aspect of the present invention to provide the method as described above, wherein the target substance is an L-amino acid.

The microalga of the present invention accumulates starch in the algae bodies at a high content. According to an exemplary embodiment, the microalga of the present invention does not need any special culture conditions such as a nitrogen-limited medium for growth and accumulation of starch, and does not need vitamin for growth.

The microalga of the present invention is useful as a source of starch for the production of glucose, which is used as a carbon source for fermentation and so forth. Moreover, the produced glucose is useful as a carbon source used for production of a target substance such as an L-amino acid by fermentation, and so forth.

### BRIEF DESCRIPTION OF THE DRAWINGS

FIG. 1 shows a phylogenetic tree of the microalga of the present invention and closely related microalgae.

FIG. 2 shows concentrations of glucose produced by reacting glucoamylase with a microalga suspension subjected to a hydrothermal treatment or a supernatant thereof.

### DESCRIPTION OF THE PREFERRED EMBODIMENTS

### <1> Microalga of the Presently Disclosed Subject Matter

The microalga of the presently disclosed subject matter belongs to the class Chlorophyceae, the genus Desmodesmus, and accumulates 30% or more of starch in algae bodies based on dry weight of the algae bodies when it is cultured under suitable conditions.

According to a phylogenetic tree created on the basis of sequence analysis of 18S rDNA, the microalga of the presently disclosed subject matter was identified to closely relate to microalgae belonging to the genus Desmodesmus such as Desmodesmus communis, Desmodesmus pirkollei and Desmodesmus costatogranulatus, and belong to the genus Desmodesmus. However, there is still some possibility that the microalga of the presently disclosed subject matter may be reclassified into another known genus or unknown genus to be newly found in future, and the expression of “microalga which belongs to the genus Desmodesmus” means that the microalga of the presently disclosed subject matter can include microalgae closely relating to those of the genus Desmodesmus according to phylogenetic classification based on sequence analysis of 18S rDNA. The genus Desmodesmus and the genus Scenedesmus having the same morphology are generally considered to be identical to each other.

According to an exemplary embodiment, the microalga of the presently disclosed subject matter can proliferate, when it is cultured in a medium not containing a vitamin. However, the microalga of the presently disclosed subject matter can be a microalga that cannot proliferate, when it is cultured in a medium not containing vitamin.

According to an embodiment, the microalga of the presently disclosed subject matter can accumulate 30% or more of starch in algae bodies based on dry weight of the algae bodies when it is cultured in a nitrogen non-limited medium. However, the microalga of the presently disclosed subject matter can be a microalga that can accumulate 30% or more of starch in algae bodies based on dry weight of the algae bodies when it is cultured in a nitrogen-limited medium.

The microalga can be obtained by, for example, isolating green algae that can grow in a medium not containing a vitamin from an environmental sample such as water of river, lake or marsh, and sea, and soil, and selecting a strain that accumulates 30% or more of starch in algae bodies based on dry weight of the algae bodies when it is cultured in an appropriate medium such as a nitrogen non-limited medium. Whether the obtained strain belongs to the genus Desmodesmus can be confirmed by creating a phylogenetic tree on the basis of sequence analysis of 18S rDNA.

Examples of the nitrogen non-limited medium include, for example, the 0.2×Gamborg's B5 medium containing 0.5 g/L or more of KNO₃ as a nitrogen source.

Specific examples of the microalga of the presently disclosed subject matter include the S-1, S-2 and S-3 strains described in the examples. These strains are designated AJ7835, AJ7838 and AJ7840, and were deposited on Apr. 12, 2010 at the Agency of Industrial Science and Technology, International Patent Organism Depository, and assigned accession numbers of FERM BP-11364, FERM BP-11365 and FERM BP-11366, respectively.

As shown in the examples, the S-1, S-2 and S-3 strains showed a starch accumulation rate of 30% or higher when they were cultured at 25° C. or 30° C. for one week in the 0.2×Gamborg's B5 medium. The S-4 strain showed a starch accumulation rate of 30% when it was cultured at 30° C. for one week in the same medium.

The suitable conditions can mean conditions that allow for a high accumulation amount of starch based on dry weight of the algae bodies. The suitable conditions can be determined by culturing the microalga and varying, for example, kind of medium, pH of medium, culture temperature, culture time, wavelength of irradiated light, exposure dose, aeration condition, and so forth, and selecting such conditions that allow for a high starch accumulation amount per unit dry weight of the algae bodies.

Examples of the medium include the 0.2×Gamborg's B5 medium, BG-11 medium, and so forth. According to an exemplary embodiment, the microalga can proliferate and accumulate starch in a medium not containing vitamin, but it can be cultured in a medium containing a vitamin.

pH of the medium is, for example, 5 to 10, or 6 to 8.

Culture temperature is, for example, 15 to 40° C., 25 to 30°, or 30° C.

Culture time is, for example, 3 to 30 days, or 5 to 14 days.

Light source for irradiation is not particularly limited so long as a light source suitable for growth of the microalga is chosen, and examples include, for example, a white fluorescent lamp.

The exposure dose of light is, for example, 0 to 50,000 lux, 500 to 30,000 lux, or 1,000 to 10,000 lux, in terms of illumination at the surface of the medium.

Examples of the aeration conditions can include those corresponding to aeration of air and/or CO₂, for example, a mixed gas of air and CO₂ having a CO₂ partial pressure of 0 to 10%, or 0.5 to 5%, into the medium. Aeration volume can be, for example, 0.1 to 2 vvm (volume per volume per minute).

Specific examples of the suitable conditions include, for example, culture in the 0.2×Gamborg's B5 medium at 30° for one week, with irradiation of light at about 4,000 lux from a white fluorescent lamp as a light source and blowing a mixed gas of air and CO₂ of which CO₂ concentration is maintained to be 3% in a volume of 500 ml/minute into the medium.

The amount of accumulated starch can be measured by, for example, disrupting the algae bodies, hydrolyzing the starch with an acid, an alkali or amylase, and measuring the produced glucose.

### <2> Method for Producing Glucose

Glucose can be produced by hydrolyzing the starch accumulated by the microalga.

Algae bodies of the microalga can be obtained by culture in the same manner as described above. The algae bodies can be collected from a culture medium by known methods, such as centrifugation, filtration, gravitational precipitation using a flocculant, or the like (Grima, E. M. et al., Biotechnol. Advances, 20:491-515, 2003).

The algae bodies can be disrupted before hydrolysis of the starch. The algae bodies can be disrupted by any method, so long as the algae bodies are sufficiently disrupted. For example, a high temperature treatment (for example, a temperature of 100° C. or higher, 150° C. or higher, 175 to 215° C., or 195 to 215° C.), an organic solvent treatment (for example, a treatment with a mixed solvent of methanol and chloroform), a boiling treatment, a strong alkali treatment, ultrasonication, French press treatment, and so forth, as well as arbitrary combinations of these can be used. The high temperature treatment includes a high temperature and high pressure reaction under the conditions for a reaction called hydrothermal reaction. If a hydrothermal reaction is performed at a high temperature, for example, 195° C. or higher, starch is fragmented, and water-soluble fractions are increased. The algae bodies can be disrupted by a physical method, after they are dried.

Although the disrupted alga can be used as it is for the hydrolysis reaction, insoluble matters such as cell walls can be removed by filtration, centrifugation, or the like, or it can also be concentrated by lyophilization or the like. Furthermore, a solution containing starch subjected to fractionation to a certain degree can also be used. For fractionation of starch from of the disrupted algae bodies, protein fractions can be separated and collected on the basis of difference in specific gravity, for example, precipitation rate in a suspension etc.

Starch can be hydrolyzed with an acid, an alkali or an enzyme such as amylase.

Starch is a high molecular weight polysaccharide consisting of amylose consisting of glucose residues linearly linked by α-1,4-glycoside linkages and amylopectin consisting of glucose residues linearly linked by α-1,4-glycoside linkages and branching by α-1,6-glycoside linkages. Amylase is a generic name of enzymes that hydrolyze glycoside linkages of starch etc. According to the difference in the action site, they are roughly classified into α-amylase (EC 3.2.1.1), β-amylase (EC 3.2.1.2) and glucoamylase (EC 3.2.1.3). α-Amylase is an endo-type enzyme which randomly cleaves α-1,4-glycoside linkages of starch, glycogen, and so forth. β-Amylase is an exo-type enzyme which cleaves α-1,4-glycoside linkage to excise maltose units one by one from the non-reducing end of starch. The glucoamylase (also called amyloglucosidase) is an exo-type enzyme which cleaves α-1,4-glycoside linkages to excise glucose units one by one from the non-reducing end of starch, and also cleaves α-1,6-glycoside linkages contained in amylopectin. Since glucoamylase produces glucose directly from starch, it is widely used for the production of glucose, and it can be used for the presently disclosed subject matter.

There are many examples of saccharification reactions of starch derived from grains, which have also been industrially implemented (Robertson, G. H. et al., J. Agric. Food Chem., 54:353-365, 2006). In the same manner as those used in these examples, a saccharification product can be obtained from algae bodies by an enzymatic reaction. When a solution containing disrupted algae bodies is subjected to an enzyme treatment, a pretreatment of boiling, ultrasonication, an alkaline treatment, and so forth in combination can be used (Izumo A. et al., Plant Science, 172:1138-1147, 2007).

Conditions of the enzymatic reaction can be suitably determined according to the characteristics of the chosen enzyme. For example, for amyloglucosidase (Sigma Aldrich, A-9228), an enzyme concentration of 2 to 20 U/mL, a temperature of 40 to 60° C., and pH 4 to 6 can be exemplified. If an organic acid that can be assimilated by a bacterium used for the production of a target substance such as L-amino acids is used for adjusting pH as a buffer, the organic acid can be used as a carbon source together with the saccharification product of starch. For example, the enzyme reaction product as it is can be added to the medium.

When starch is hydrolyzed, an oligosaccharide such as maltose can be produced in addition to glucose. Glucose produced from starch derived from the microalgae can contain such an oligosaccharide.

Furthermore, glucose produced by the method of the presently disclosed subject matter can contain a carbohydrate other than starch produced by the microalga, saccharified product thereof, fats and oils, decomposition product thereof, and so forth.

Hydrolysate of starch containing glucose can be used as it is, or can also be used as a dried product after removing moisture depending on the use. Glucose can also be roughly or fully purified.

### <3> Method for Producing a Target Substance

Glucose obtained by the aforementioned method can be used as, for example, a carbon source for production of a target substance by fermentation.

Production of an L-amino acid using a microalga, in which a culture of the algae is processed at a moderate temperature, a supernatant containing glucose is obtained by centrifugation, and L-amino acid-containing medium is collected has been reported (WO2011/013707). According to this method, glucose can be produced from a microalga without using amylase, or with only using a small amount of amylase. This method can also be applied to the microalga of the presently disclosed subject matter.

The target substance to be produced is not particularly limited, so long as it is a substance that can be produced by a microorganism using glucose as a carbon source, and examples include amino acids, nucleic acids, vitamins, antibiotics, growth factors, physiologically active substances, proteins, and so forth. These target substances can be in the form of a salt.

Examples of the amino acids include L-glutamic acid, L-glutamine, L-lysine, L-leucine, L-isoleucine, L-valine, L-tryptophan, L-phenylalanine, L-tyrosine, L-threonine, L-methionine, L-cysteine, L-cystine, L-arginine, L-serine, L-proline, L-asparatic acid, L-asparagine, L-histidine, glycine, L-alanine, and so forth. The amino acids can be amino acids in free form, or in the form of a salt such as sulfate, hydrochloride, carbonate, ammonium salt, sodium salt and potassium salt.

Examples of the nucleic acids include inosine, guanosine, xanthosine, adenosine, inosinic acid, guanylic acid, xanthylic acid, adenylic acid, and so forth. The nucleic acids can by a nucleic acid in free form, or can be in the form of a salt such as sodium salt and potassium salt.

The microorganism used for the presently disclosed subject matter is not particularly limited, so long as the chosen microorganism can produce a target substance using glucose as a carbon source, and examples include enterobacteria belonging to γ-Proteobacteria such as those of the genera Escherichia, Enterobacter, Pantoea, Klebsiella, Raoultella, Serratia, Erwinia, Salmonella, and Morganella, so-called coryneform bacteria such as those belonging to the genus Brevibacterium, Corynebacterium, or Microbacterium, bacteria such as those belonging to the genus Alicyclobacillus or Bacillus, yeasts belonging to the genus Saccharomyces or Candida, and so forth.

L-Amino acid-producing bacteria, nucleic acid-producing bacteria, microorganisms used for breeding thereof, and methods for imparting or enhancing an L-amino acid-producing ability or nucleic acid-producing ability are described in detail in WO2007/125954, WO2005/095627, U.S. Patent Published Application No. 2004/0166575, and so forth.

The microorganism can be cultured in the same manner as for a typical fermentation, except that glucose derived from microalga is used as a carbon source. As a culture vessel, usual culture apparatuses such as a fermentation tank or fermenter can be used.

As for the medium, a media typically used for the production of a target substance using a microorganism, specifically, a medium containing a carbon source, a nitrogen source, and inorganic salts as well as other organic micronutrients, such as amino acids and vitamins, as required, can be chosen. Either a synthetic medium or a natural medium can be used.

The carbon source contained in the medium can consist of glucose alone, or can consist of a mixture of glucose and another carbon source. Examples of the other carbon source include glycerol, saccharides such as fructose, maltose, mannose, galactose, starch hydrolysate, and molasses, organic acids such as acetic acid and citric acid, and alcohols such as ethanol.

As the nitrogen source, ammonia, ammonium salts such as ammonium sulfate, ammonium carbonate, ammonium chloride, ammonium phosphate, and ammonium acetate, nitrates, and so forth can be used.

As the organic micronutrients, amino acids, vitamins, aliphatic acids, and nucleic acids, as well as peptone, casamino acid, yeast extract, soybean protein degradation product and so forth containing the foregoing substances can be used. When an auxotrophic mutant strain that requires an amino acid or the like for growth thereof is used, the required nutrient can be supplemented to the medium.

As the inorganic salts, phosphoric acid salts, magnesium salts, calcium salts, iron salts, manganese salts, and so forth can be used.

The culture conditions can be appropriately determined according to the microorganism to be used.

As for the method of collecting a target substance from the culture medium after completion of culture, the target substance can be collected by any known collection method according to the type of the target substance. For example, when the target substance is an amino acid, the target substance is collected by a method of removing cells from culture medium, and then concentrating the medium to crystallize the target substance, ion exchange chromatography, or the like.

For the collection of the target substance from culture medium after completion of the culture, no special method is required.

The target substance collected according to the presently disclosed subject matter can contain microbial cells, medium components, moisture, and microbial metabolic by-products, in addition to the target substance.

### EXAMPLES

Hereafter, the present invention will be more specifically explained with reference to the following non-limiting examples.

### Example 1

### Acquisition of Microalgae Strains that Highly Accumulate Starch

(1) Culture of Water or Soil Samples

Samples of water or soil were collected from ponds, rivers, paddy fields in various parts of Japan.

To 10 ml of the 0.2×Gamborg's B5 medium (NIHON PHARMACEUTICAL) contained in a 50 ml-volume conical flask, a small amount of water sample or soil sample was added, and ampicillin and streptomycin were further added as antibiotics at a final concentration for each antibiotic of 100 ppm. Culture was performed with shaking each flask on a plant incubator CL-301 (TOMY). After two weeks from the start of the culture, proliferation of green algae could be visually confirmed. As for the culture conditions, the CO₂ concentration in the incubator was controlled to be about 3% by aeration of a mixed gas of air and CO₂ in the plant incubator using a portable gas mixing apparatus PMG-1 (Kofloc). The inside of the plant incubator was continuously irradiated with a white fluorescent lamp as a light source (illumination: about 4,000 lux), and the temperature was maintained at 30° C. The composition of the Gamborg's B5 medium is as follows.

Composition of 1×Gamborg's B5 medium (NIHON PHARMACEUTICAL)

| | | |
|---|---|---|
| KNO₃ | 2500 | mg |
| MgSO₄•7H₂O | 250 | mg |
| NaH₂PO₄•H₂O | 150 | mg |
| CaCl₂•2H₂O | 150 | mg |
| (NH₄)₂SO₄ | 134 | mg |
| Na₂•EDTA | 37.3 | mg |
| FeSO₄•7H₂O | 27.8 | mg |
| MnSO₄•H₂O | 10 | mg |
| H₃BO₃ | 3 | mg |
| ZnSO₄•7H₂O | 2 | mg |
| KI | 0.75 | mg |
| Na₂MoO₄•2H₂O | 0.25 | mg |
| CuSO₄•5H₂O | 0.025 | mg |
| CoCl₂•6H₂O | 0.025 | mg |
| Distilled water | 1000 | ml |

(2) Isolation of Green Algae

Agarose was added to the 0.2×Gamborg's B5 medium at a final concentration of 1.5%, and the medium was sterilized by autoclaving (120° C., 15 minutes), and then poured into petri dishes in a volume of 30 ml per dish to prepare plate medium of the 0.2×Gamborg's B5 medium.

The culture medium in which proliferation of green algae could be confirmed in the foregoing section was plated on the plate medium of the 0.2×Gamborg's B5 medium, and culture was performed for 2 weeks under the same conditions as those mentioned above, except that shaking was not performed. When preferential proliferation of contaminant bacteria was observed on the plate medium, sterilization of the culture medium was performed with a hypochlorite treatment. Specifically, a sodium hypochlorite solution having an effective chlorine concentration of 8.5 to 17.5% was diluted 100 times with sterilized water, the diluted solution was mixed with the culture medium so as to obtain an effective chlorine concentration of 100 ppm, and the mixture was left to stand at room temperature for 10 minutes. Then, a sodium thiosulfate solution with an adjusted concentration of 1,000 ppm was added to the medium so that the thiosulfate concentration was 10 times the effective chlorine concentration, the medium was applied to the plate medium of the 0.2×Gamborg's B5 medium, and culture was performed for 2 weeks. Single colonies were collected with a platinum loop from the plates on which favorable proliferation of green algae could be confirmed, and applied to the plate medium of the 0.2×Gamborg's B5 medium, and culture was further performed for two weeks to obtain isolated strains of algae. The five strains obtained as described above were designated S-1, S-2, S-3, S-4 and S-5 strains.

(3) Molecular Phylogenetic Analysis of Isolated Green Alga Strains

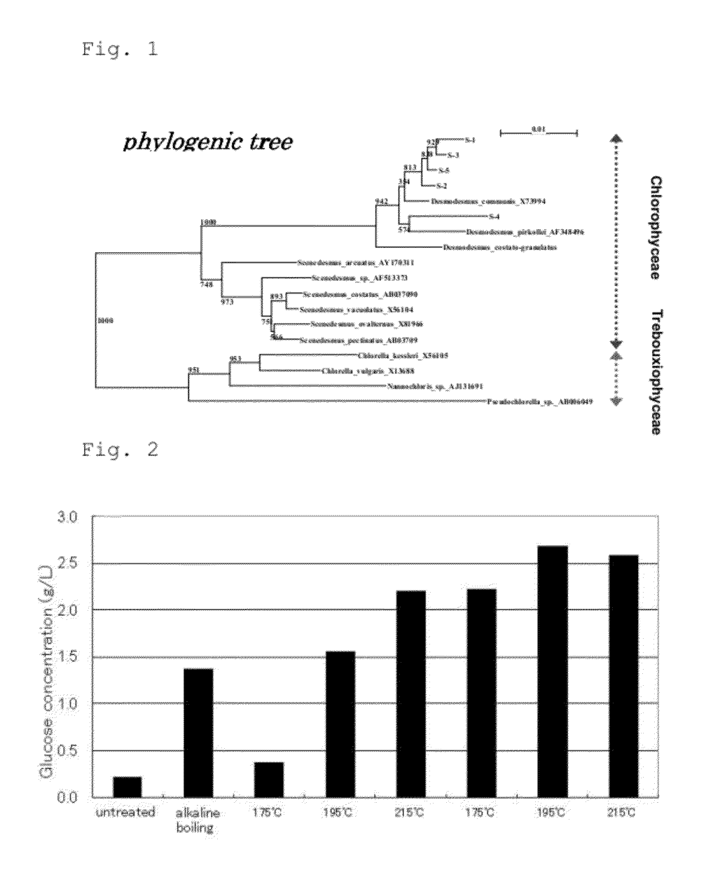

Molecular phylogenetic analysis of the green alga strains isolated as described above was performed on the basis of 18S rDNA sequence as an index by using universal primers for amplification of 18S rDNA region of green algae (primer set 1: SEQ ID NOS: 1 and 2, primer set 2: SEQ ID NOS: 3 and 4). The determined 18S rDNA region sequences of the S-1, S-2, S-3, S-4 and S-5 strains are shown in SEQ ID NOS: 5 to 9, respectively. For these sequences, BLAST search was performed in the NCBI database (http://www.ncbi.nlm.nih.gov/Blast.cgi) to obtain data of highly homologous 18S rDNA sequences derived from green algae and create a phylogenetic tree. Clustal X2 was used for multiple alignment, Sea View for edition, and NJplot for display and edition of the phylogenetic tree. The phylogenetic tree was created according to the neighbor-joining method of Clustal X2, with the random number for bootstrap of 111 and number of times of bootstrap of 1000. The obtained phylogenetic tree is shown in FIG. 1. It became clear from the result that the S-1, S-2, S-3, S-4 and S-5 strains are closely related to the genus Desmodesmus.

(4) Measurement of Starch Amount

A colony of each isolated green alga strain on the plate medium collected with a platinum loop was transferred into 10 ml of the 0.2×Gamborg's B5 medium contained in a 50-ml volume conical flask, and culture was performed for one week. This culture medium (200 μl) was added to 10 ml of fresh 0.2×Gamborg's B5 medium contained in a flask, the inside of the plant incubator was filled with a mixed gas of air and CO₂ of which CO₂ concentration was maintained to be 3%, culture was performed for one week under continuous irradiation at an illumination of 8,000 lux, and then amount of starch was measured. The culture was performed at two different culture temperatures, 25° C. and 30° C.

The amount of starch was measured as follows. Each culture medium of green alga (1 ml) was put into a 1.5-ml volume tube, and centrifuged (12,000 rpm, 10 minutes), and then the supernatant was removed. Then, ethanol (1 ml) was added to the alga body residue to suspend it, and the suspension was subjected to a boiling treatment (95° C., 30 minutes). The sample subjected to the treatment was centrifuged, the supernatant was removed, and the obtained precipitates were dried for 5 minutes with a centrifugal concentrator PV-1200 (WAKENYAKU). Then, 1 ml of 0.2 M KOH was added to the precipitates to suspend them, and the suspension was subjected to a boiling treatment (95° C., 30 minutes) to perform alkali hydrolysis of the starch components derived from the algae bodies. The pH of the solution obtained by the alkali hydrolysis was adjusted to about 5.5 by adding 200 μl of 1 M CH₃COOH. Amyloglucosidase (2 unit, Sigma-Aldrich, A-9228) was added to the solution, the tube was set on a tube rotator, and the reaction was allowed in an incubator at 55° C. for 24 hours.

The obtained reaction mixture was centrifuged, then the glucose concentration in the obtained supernatant was measured with Biotech Analyzer AS210 (Sakura Seiki), and the amount of starch was calculated. Furthermore, 1 ml of the culture medium of the green alga was put into a 1.5 ml-volume tube, and centrifuged (14,000 rpm, 5 minutes), the supernatant was removed, then the residue was dried at 55° C. for 24 hours, and dry alga body weight was measured. In addition, the amount of starch per unit dry alga body weight was calculated as the starch accumulation rate. The results are shown in Table 1.

TABLE 1

Starch accumulation rate

| Strain | 25° C. | 30° C. |
|---|---|---|
| S-1 | 36% | 37% |
| S-2 | 31% | 35% |
| S-3 | 32% | 30% |
| S-4 | 25% | 30% |
| S-5 | 4% | 2% |

The S-1, S-2 and S-3 strains showed a starch accumulation rate of 30% or higher for both culture temperatures of 25° C. and 30° C. The S-4 strain showed a starch accumulation rate of 30% for the culture temperature of 30° C.

### Example 2

### Preparation of Glucose from Starch Derived from S1 Strain

Culture medium (30 ml) of the S-1 strain cultured in the same manner as described above was added to 1500 ml of the 0.2×Gamborg's B5 medium contained in a 2 L-volume culture tank (ABLE), the tank was set on a light irradiation type S-jar culture apparatus (Ishikawa Seisakusho), and culture was performed for seven days under the conditions of 30° C. and light intensity of 20,000 lux with shaking and blowing a mixed gas of air and CO₂ having a CO₂ concentration of 3% into the medium at a rate of 500 ml/minute. From this culture medium of the S-1 strain (6 L), 20-fold concentrate (300 ml) was prepared by centrifugation and resuspension in water, the concentrate was put into a vessel for a hydrothermal reaction apparatus (OM Lab-Tech, MMJ-500), heated to 195° C. over 40 minutes with shaking, maintained at 195° C. for 5 minutes, and then rapidly cooled to prepare a hydrothermal treatment product. The dry alga body weight per 1 L of the algae culture medium was 3 to 4 g/L.

Then, the entire hydrothermal treatment product was transferred to a 500 ml-volume jar vessel (ABLE), and adjusted to a reaction temperature of 55° C., 6000 units of amyloglycosidase (Sigma-Aldrich, A-9228) sterilized by filter sterilization was added to the product, and the reaction was allowed for 24 hours with shaking at 400 rpm. Then, the saccharification reaction solution was filtered with qualitative filter paper (ADVANTEC), and the filtrate was adjusted to pH 7.0 with a 1 N NaOH solution, and then sterilized by autoclaving (115° C., 10 minutes) to obtain glucose derived from green alga. As a result, the concentration of glucose derived from green alga after the saccharification was 30.8 g/L.

### Example 3

### Examination of Fragmentation of Starch Derived from S1 Strain

An alga body concentrate of the S-1 strain was subjected to a hydrothermal treatment in the same manner as that of Example 2, except that the heating temperature was 175° C., 195° C. or 215° C. A sufficient amount of amyloglycosidase was added to the hydrothermal treatment product or supernatant thereof obtained by centrifugation, and the reaction was allowed at 55° C. for 16 hours. Then, the amount of generated glucose was measured.

Furthermore, 0.2 M KOH was added to the alga body concentrate of the S-1 strain, and the reaction was allowed at 95° C. for 30 minutes to perform alkali hydrolysis. The reaction mixture was adjusted to pH 5.5 by adding 1 M acetic acid, then a sufficient amount of amyloglycosidase was added to the mixture, and the reaction was allowed at 55° C. for 16 hours. Then, the amount of generated glucose was measured.

The results are shown in FIG. 2. From these results, it was revealed that the starch in the microalga became more fragmented as the temperature of the hydrothermal treatment increased.

### Example 4

### L-Glutamic Acid Production Culture

As an L-glutamic acid-producing bacterium, the Corynebacterium glutamicum ΔS strain (WO95/34672, U.S. Pat. No. 5,977,331) was used. The ΔS strain is a strain obtained by disrupting the sucA (odhA) gene coding for the E1o subunit of α-ketoglutarate dehydrogenase of a Corynebacterium glutamicum wild-type strain (ATCC 13869).

The ΔS strain was inoculated on the CM-Dex plate medium, and cultured at 31.5° C. for 24 hours. The cells on the plate medium were scraped up in an amount of one platinum loop, inoculated in 20 mL of an L-glutamic acid production medium having the following composition contained in a Sakaguchi flask, and cultured at a culture temperature of 31.5° C. for 24 hours. Culture was performed by using, as a carbon source for the main culture, a saccharification solution prepared from the alga starch degradation product of the S-1 strain (containing 30.8 g/L of glucose and 0.81 g/L of glycerol), or reagent glucose of substantially the same concentration for control.

Composition of L-Glutamic Acid Production Medium

(Group A)

| | | |
|---|---|---|
| Carbon source | 19.4 | g/L |
| Alga starch degradation product (containing 19.1 g/L of | | |
| glucose and 0.5 g/L of glycerol as final concentrations) or | | |
| Reagent glucose | | |

(Group B)

| | | |
|---|---|---|
| (NH₄)₂SO₄ | 15 | g/L |
| KH₂PO₄ | 1 | g/L |
| MgSO₄•7H₂O | 0.4 | g/L |
| FeSO₄•7H₂O | 10 | mg/L |
| MnSO₄•4H₂O | 10 | mg/L |
| VB1•HCl | 200 | μg/L |
| Biotin | 300 | μg/L |
| Soybean hydrolysate | 0.48 | g/L |

(Group C)

| | | |
|---|---|---|
| Calcium carbonate | 50 | g/L |

The components of Groups A and B were adjusted to pH 7.8 and pH 8.0, respectively, with KOH, and sterilized by autoclaving at 115° C. for 10 minutes, and the component of Group C was subjected to hot air sterilization at 180° C. for 3 hours. After the components of the three groups were cooled to room temperature, they were mixed.

After completion of the culture, the amount of the accumulated L-glutamic acid was measured with Biotech Analyzer AS210 (Sakura Seiki). Furthermore, since L-glutamic acid derived from the soybean hydrolysate was contained in the L-glutamic acid production medium, the values obtained by subtracting the L-glutamic acid amount in the soybean hydrolysate among the medium components from the measured values are shown in Table 2. From the results obtained after the culture for 24 hours, it was found that the amount of accumulated L-glutamic acid was improved as compared to that obtained by using the reagent glucose. These results demonstrated that starch degradation product derived from green alga was useful as a carbon source for L-glutamic acid production culture.

TABLE 2

| Carbon source | L-Glutamic acid concentration (g/L) |
|---|---|
| Reagent glucose 19.4 g/L | 11.3 |
| Alga glucose 19.1 g/L | 12.4 |

The results of total organic carbon (TOC) analysis performed for the saccharification solution prepared from the aforementioned alga starch degradation product of the S-1 strain and the reagent glucose are shown in Table 3.

TABLE 3

| | Algae glucose | Reagent glucose |
|---|---|---|
| TOC (mol/L) | 1.03 | 0.83 |
| Glu (mol/L) | 0.42 | 0.39 |
| Glu/TOC | 41% | 47% |

TOC of the saccharification solution derived from the S-1 strain was higher than that of the reagent glucose, and the glucose amount relative to TOC was higher in the reagent glucose. From these results, it is estimated that glucose contained in the saccharification solution derived from the S-1 strain partially included glucose derived from a carbon source other than starch.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A microalga which belongs to the genus Desmodesmus and accumulates 30% or more of starch in algae bodies based on dry weight of the algae bodies when the microalga is cultured under suitable conditions.

2. The microalga according to claim 1, which can proliferate in a medium not containing vitamin.

3. The microalga according to claim 1, which accumulates 30% or more of starch in the algae bodies based on dry weight of the algae bodies when microalga is cultured in a nitrogen non-limited medium.

4. The microalga according to claim 1, which accumulates 30% or more of starch in algae bodies based on dry weight of the algae bodies when the microalga is cultured at 30° C. for one week in 0.2×Gamborg's B5 medium.

5. The microalga according to claim 1, which is selected from the group consisting of the strains AJ7835 (FERM BP-11364), AJ7838 (FERM BP-11365) and AJ7840 (FERM BP-11366).

6. A method for producing glucose, which comprises hydrolyzing starch accumulated in the microalga according to claim 1 to produce glucose.

7. A method for producing a target substance, which comprises culturing a microorganism that produces the target substance in a medium containing glucose produced by the method according to claim 6, and collecting the target substance from culture.

8. The method according to claim 7, wherein the target substance is an L-amino acid.


SEQUENCE LISTING (9 sequences)

SEQ ID NO: 1 - DNA, 23 bp, Artificial Sequence
  primer
tacctggttgatcctgccagtag

SEQ ID NO: 2 - DNA, 21 bp, Artificial Sequence
  primer
ccaatccctagtcggcatcgt

SEQ ID NO: 3 - DNA, 23 bp, Artificial Sequence
  primer
agataccgtcgtagtctcaacca

SEQ ID NO: 4 - DNA, 25 bp, Artificial Sequence
  primer
accttgttacgacttctccttcctc

SEQ ID NO: 5 - DNA, 1765 bp, Desmodesmus sp.
tacctggttgatcctgccagtagtcatatgcttgtctcaaagattaagccatgcatgtctaagtataaactgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccggactttgtccgacccgcggtgaatcatgatatcttcacgaagcgcatggccttgtgccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcattttatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggtttcagcggtccgcctatggtgagcactgctgtggccttccttactgtcggggacctgcttctgggcttcgttgtccgggacagggattcggcatggttactttgagtaaattagagtgttcaaagcaggcttacgccgtgaatactttagcatggaataacacgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatggtttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcacagtcgctttttgcgggtggtttgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatgggtatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggctgttgaaggtggcaacaccgtcgattgccgagaagttcattaaaccctcccacctagaggaaggagaagtcgtaacaaggt

SEQ ID NO: 6 - DNA, 1767 bp, Desmodesmus sp.
tacctggttgatcctgccagtagtcatatgcttgtctcaaagattaagccatgcatgtctaagtataaactgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccggactttgtccgacccgcggtgaatcatgatatcttcacgaggcgcatggccttgtgccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcattttatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggtttcagcggtccgcctatggtgagcactgctgtggccttccttactgtcggggacctgcttctgggcttcattgtccgggacagggattcggcatggttactttgagtaaattagagtgttcaaagcaggcttacgccgtgaatactttagcatggaataacacgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcactgtcgctttttgcggctggcttttgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattatcggtggcaacaccgtcgattgccgagaagttcattaaaccctcccacctagaggaaggagaagtcgtaacaaggt

SEQ ID NO: 7 - DNA, 1766 bp, Desmodesmus sp.
tacctggttgatcctgccagtagtcatatgcttgtctcaaagattaagccatgcatgtctaagtataaactgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccggactttgtccgacccgcggtgaatcatgatatcttcacgaagcgcatggccttgtaccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcattttatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggtttcagcggtccgcctatggtgagcactgctgtggccttccttactgtcggggacctgcttctgggcttcattgtccgggacagggattcggcatggttactttgagtaaattagagtgttcaaagcaggcttacgccgtgaatactttagcatggaataacacgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcactgtcgctttttgcggctggctttgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgacgctcctaccgattgggtgtgctggtgaagtgttcggattggctgttgaaggtggcaacaccgtcgattgccgagaagttcattaaaccctcccacctagaggaaggagaagtcgtaacaaggt

SEQ ID NO: 8 - DNA, 2688 bp, Desmodesmus sp.
tacctggttgatcctgccagtagtcatatgcttgtctcaaagattaagccatgcatgtctaagtataaactgcttatactgtgaaactgcgaatggctcattaaatcagttatagtttatttggtggtaccttcttactcggaataaccgtaagaaaattagagctaatacgtgcgtaaatcccgactcctggaagggacgtatatattagataaaaggccgaccgggctctgcccgacccgcggtgaatcatgatatcttcacgaagcgtatggccctgcgccgacgctgttccattcaaatttctgccctatcaactttcgatggtaggatagaggcctaccatggtggtaacgggtggcggaggattagggttcgattccggagagggagcctgagaaacggctaccacatccaaggaaggcagcaggcgcgcaaattacccaatcctgatacggggaggtagtgacaataaataacaataccgggcatatcatgtctggtaattggaatgagtacaatctaaatcccttaacgaggatccattggagggcaagtctggtgaacacttcgacacatgctgttgacgccagagatagtagggcagcctcagggctgttatgcctgctagtcgagcaaccatcgactgtggaatgggttggttgccggcaagacgacctggtacggggaaggctaagttgcagtgtgatgcaatatgctaatcccgtggcgagctcgcaaagggtgacttttgcacagccgtcgtaacgcacggaaaggcgtcggctgactctctgtcagagagttggcttaagggacgtgctaaccccatccgaaaggatgctcaaagcaacagcacccattctgcaaaggctttgaggagcaacagtgtgatgaggaaatgcttcacactgctcggtagtaaggcattggattcgccaggacaggatccagccaagtggccagcagccgcggtaattccagctccaatagcgtatatttaagttgttgcagttaaaaagctcgtagttggatttcgggtgggtttcagcggtccgcctatggtgagtactgctgtggcctatcttactgtcggagacctgcttctgggcttcattgtccgggacaggggttcggcatggttactttgagtaaattagagtgttcaaagcaggcttacgccgtgaatactttagcatggaataacactataggactctgccctattgtgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgttaatgctgaaagctctagcgccagtgagagggaaacctcttgctagtcagagagcagtgatgttcgcggttgctgctcactggcgacaccttcaaattgctgggaaatcctaaagccgatgcataccaaagcatggtggaaacaccgtgccggccagggtaattacctcgggtatggtgacaactgcattggatgaggtgaggtcactcaccaaaatggacaaccagcagccaagtcctaagggcacaacacgtgcctatggatgcagttcacagactaaatggaggtgggctgtcacgcaaggtatacgtccaatatcggcaatgcttggacggtccgatgccttggaccgatcgaaccttgcgcgatggcctaagatatagtcggtccttatcgaaagataacctgcaagaggatatcaagtgcgaaacttgattgagagcttgcaggacaccggagtagtacttgacagtgcaaactgctaagggcaccacagcggtggagcctctggtgtgggcacaacgggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcactgtcgctttttgcggctggctttgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgactacgtccctgccctttgtacacaccgcccgtcgctcctaccgattgggtgtgctggtgaagtgttcggattggcaactgaaggtggcaacaccgtcggttgccgaaaagttcattaaaccctcccacctagaggaaggagaagtcgtaacaaggt

SEQ ID NO: 9 - DNA, 1766 bp, Desmodesmus sp.
tacctggttgatcctgccagtagtcatatgcttgtctcaaagattaagccatgcatgtctaagtataaactgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccggactttgtccgacccgcggtgaatcatgatatcttcacgaagcgcatggccttgtgccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcattttatgtctggtaattggaatgagtacaatctaaatcccttaacgagggtccattggagggcaagtctggtgccagcagccgcggtaattccagctccaatagcgtatatttaagttgttgcagttaaaaagctcgtagttggatttcgggtgggtttcagcggtccgcctatggtgagcactgctgtggccttccttactgtcggggacctgcttctgggcttcattgtccgggacagggattcggcatggttactttgagtaaattagagtgttcaaagcaggcttacgccgtgaatactttagcatggaataacacgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcactgtcgctttttgcggctggctttgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattgaaggtggcaacaccgtcgattgccgaaaagttcattaaaccctcccacctagaggaaggagaagtcgtaacaaggt